(19) United States
(12) Patent Application Publication
(10) Pub. No.: US 2022/0204470 A1
(43) Pub. Date: Jun. 30, 2022

(54) APPARATUS FOR AND METHOD OF CONVERTING CBD AND/OR CBD DERIVATIVES TO AT LEAST ONE OTHER TYPE OF CANNABINOID AND/OR CANNABINOID DERIVATIVE SUCH AS THC

(71) Applicant: RAPID DOSE THERAPEUTICS CORP., Burlington (CA)

(72) Inventors: Jason Lewis, Burlington (CA); Benjamin James Macphail, Burlington (CA); James Mcnulty, Burlington (CA); Alexander James Nielsen, Burlington (CA); Rina Carlini, Burlington (CA)

(21) Appl. No.: 17/601,636

(22) PCT Filed: Apr. 3, 2020
(86) PCT No.: PCT/CA2020/050445

§ 371 Date: Oct. 5, 2021

Related U.S. Application Data
(60) Provisional application No. 62/830,350, filed on Apr. 5, 2019.

(51) Int. Cl. C07D 311/80; B01J 21/16; B01J 39/05; B01J 47/02

(52) U.S. Cl. CPC C07D 311/80; B01J 47/02; B01J 39/05; B01J 21/16

(57) ABSTRACT

The specification relates to a process for preparation of a compound of Formula (II), the process involving the step of reacting a compound of Formula (I), in a solvent, in the presence of a solid supported acid catalyst to form the compound of Formula (II), where R¹, R², R³, R⁴, R⁵ and [symbol] are as described herein.

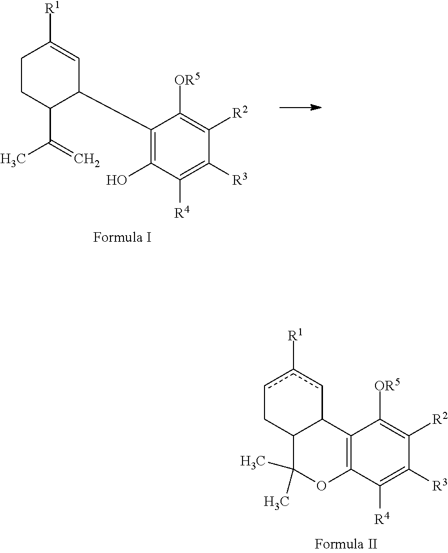

### CROSS-REFERENCE TO RELATED APPLICATIONS

This application claims the benefit of and priority to U.S. Provisional Patent Application No. U.S. 62/830,350, filed Apr. 5, 2019 under the title APPARATUS FOR AND METHOD OF CONVERTING CBD AND/OR CBD DERIVATIVES TO AT LEAST ONE OTHER TYPE OF CANNABINOID AND/OR CANNABINOID DERIVATIVE SUCH AS THC. The content of the above patent application is hereby expressly incorporated by reference into the detailed description hereof.

### FIELD

The specification relates to the chemical synthesis of cannabinoids and/or cannabinoid derivatives. In a particular aspect, the specification relates to converting CBD (cannabidiol) and/or CBD derivatives to at least one other type of cannabinoid and/or cannabinoid derivative. In another aspect, the specification relates to an apparatus for and methods of converting CBD and/or CBD derivatives to at least one other type of cannabinoid and/or cannabinoid derivative.

### BACKGROUND

Cannabis refers to materials, compounds and extracts derived from the plants of the Cannabis genera, which are a member of the Cannabaceae angiosperm plant family. These materials include raw and dried plant, extracts, resins, metabolites, compounds, distillates and other processed materials derived from the plant. While almost 600 unique secondary metabolites or compounds have been identified in cannabis (Lewis et al, ACS Omega, 2017, 2, 6091-6103, incorporated herein by reference), just over 100 of these are terpenophenolic phytocannabinoids (Welling et al, Front. Plant Sci. 2018, 9, 1510, incorporated herein by reference). Several of these phytocannabinoid metabolites have proven of value in medicinal chemistry, for example CBD was FDA approved (2018) under the trade name Epidiolex for the treatment of Lennox-Gastaut syndrome and Dravet's syndrome, two forms of epilepsy. In addition, Δ⁹-THC (Δ⁹-Tetrahydrocannabinol) is FDA approved under the trade names Marinol and Syndros (generic name dronabinol) for the treatment of anorexia and chemotherapy associated nausea and vomiting.

Many other potential indications for cannabis are under investigation (Halford, B. Chem. & Eng. News, Jul. 23, 2018, pp 28-33, incorporated herein by reference), including natural extracts and resins, purified individual metabolites and total and semi-synthetic versions thereof (McCoy, M, Chem & Eng. News, Nov. 19 2018, pp. 20-21, incorporated herein by reference). While phytocannabinoids such as CBD are generally deemed non-psychoactive (Grotenhermen et al, Cannabis and Cannabinoid Res., 2017, v. 2.1, p. 1, incorporated herein by reference), derivatives such as Δ⁸-THC and Δ⁹-THC are considered potent psychoactive components. The demonstrated uses and studies on the potential application of natural, synthetic and semi-synthetic phytocannabinoids as human pharmaceuticals, veterinary products and other activities constitutes a rapidly expanding area of research (Hill, K. P., JAMA, 2015, 313, 2474-2483; Whiting et al, JAMA, 2015, 313, 2456-2473; Welty, et al Epilepsy Currents, 2014, 14, 250-252, all incorporated herein by reference).

The conversion of CBD to THC derivatives including Δ⁸-THC and Δ⁹-THC has been reported using various solvents and catalysts.

The conversion of CBD to Δ⁹-THC was reported in low yield (2%) by refluxing (2 h) an ethanolic solution of CBD containing hydrogen chloride (Gaoni et al J. Am. Chem. Soc. 1964, 86, 1646, incorporated herein by reference).

The yield on the conversion of CBD to Δ⁹-THC was subsequently improved (70% reported) using boron trifluoride as catalyst (Gaoni et al J. Am. Chem. Soc. 1971, 93, 217-224, incorporated herein by reference).

Gaoni et al (Tetrahedron, 1966, 22, 1481-1488, incorporated herein by reference) described a method to convert CBD to a mixture of cannabinoids, including, Δ⁸-THC and Δ⁹-THC, by refluxing (18 h) a solution containing CBD in ethanol using hydrochloric acid, followed by extraction and chromatographic purification, yielding both Δ⁸-THC and Δ⁹-THC. In another variation of this method, a solution of CBD in benzene containing p-toluenesulfonic acid was refluxed (2 h) and after extractive work-up, purification and distillation, gave Δ⁸-THC in 64% reported yield.

In addition, U.S. Pat. Appl. No. 2004/0143126 A1 (incorporated herein by reference), describes the conversion of CBD to Δ⁸-THC and Δ⁹-THC employing a range of soluble acidic catalysts such as boron trifluoride, boron trifluoride diethyl etherate or p-toluenesulfonic acid.

U.S. Pat. Appl. No. 2004/0143126 A1 (incorporated herein by reference), describes the conversion of CBD to Δ⁸-THC with some selectivity by refluxing (1 h) a solution of CBD in toluene containing p-toluenesulfonic acid under nitrogen. After extractive work-up and chromatographic purification, Δ⁸-THC was isolated in 81% yield.

U.S. Pat. Appl. No. 2004/0143126 A1 (incorporated herein by reference), describes the conversion of CBD to Δ⁹-THC with some selectivity by stirring (1 h) a solution of CBD in dichloromethane at 0° C. containing boron trifluoride diethyl etherate under nitrogen. After extractive work-up and chromatographic purification, Δ⁹-THC was isolated in 57% yield.

The use of a soluble Lewis acid of general formula MY where M is selected from B, Al, Sc, Ti, Yt, Zr, La, Li, Hf or Zn and Y can be selected from F, Cl, Br, I, trifluoroacetate (triflate) alkoxide and combinations thereof has been reported (Dialer et al, U.S. Pat. Appl. 2017/0008868 A1, incorporated herein by reference) in the conversion of CBD to Δ⁸-THC and Δ⁹-THC. In a particular embodiment, the use of Lewis acid catalysts such as zinc triflate or scandium triflate are shown to affect the conversion of CBD to Δ⁹-THC.

The use of solid catalysts based on natural clay materials such as Montmorillonite K 10 (MK10) has been shown (Nagano et al, Tetrahedron 1999, 55, 2591-2608, incorporated herein by reference) to activate terpenoid materials such as geraniol and oligomeric prenols. The activation of allylic alcohols was shown to lead to coupling of these materials leading to complex mixtures of oligomers.

The use of Lewis acid catalysts, including ZnBr₂, Ti(OiPr)₄, BF₃-OEt₂, TiO₂, and TiC₄ have been shown to affect condensation of a monoterpenoid derived aldehyde, such as citronellal or citral (geranial), with a methyl-resorcinol (orcinol) derivative (Giorgi et al, Eur. J. Org. Chem., 2018, 1307-1311, incorporated herein by reference). The use of these catalysts was reported to lead to the formation of truncated THC analogs with poor selectivity.

The use of solid supported materials, including zeolite, Amberlyst and montmorillonite-doped with metal cations (M-MMT), selected from Na, Li, Ge, Sn and Ti, have been shown to affect condensation of a monoterpenoid derived aldehyde, such as citronellal or citral (geranial), with a methyl-resorcinol (orcinol) derivative (Giorgi et al, Eur. J. Org. Chem., 2018, 1307-1311, incorporated herein by reference). The use of these catalysts was reported to lead to the formation of truncated THC analogs with poor selectivity. The use of M-MMTs also provides mixtures that include regioisomers such as the ortho-tetrahydrocannabinols.

Three cannabinoids can be of particular interest for medicinal and recreational uses: cannabidiol (CBD), Δ⁸-tetrahydrocannabinol (Δ⁸-THC), and Δ⁹-tetrahydrocannabinol (Δ⁹-THC) (Scheme 1).

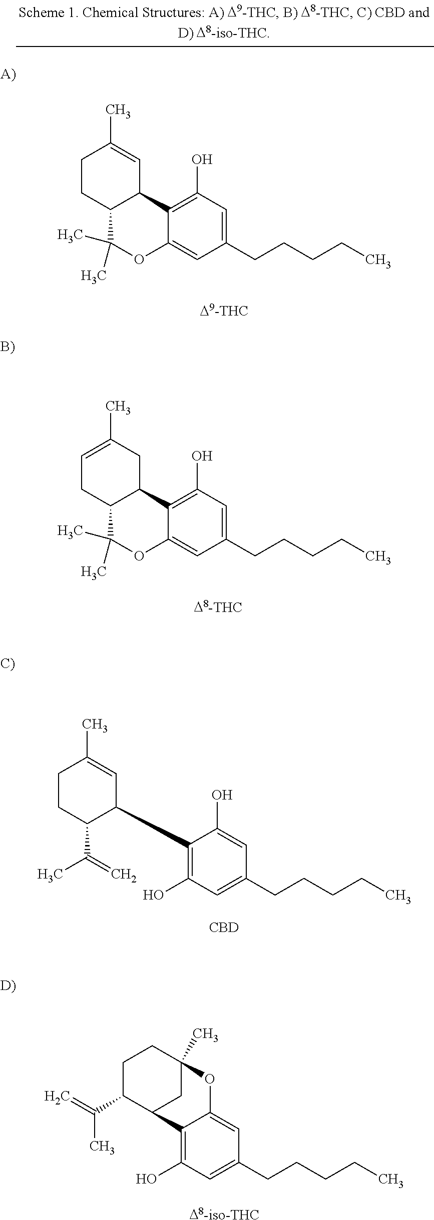

There is a need in the art for a process for conversion of CBD or a derivative thereof in to at least one other type of cannabinoid. In addition, there is a need in the art for the intra-molecular cyclization of CBD or derivative thereof to form another type of a cannabinoid. Further, there is a need in the art for a method for the process noted herein. Moreover, there is a need in the art an apparatus for carrying out the process disclosed herein.

### SUMMARY

In one aspect, the specification relates to a process for preparation of a compound of Formula II, comprising:

reacting a compound of Formula I, in a solvent, in the presence of a solid supported acid catalyst to form the compound of Formula II

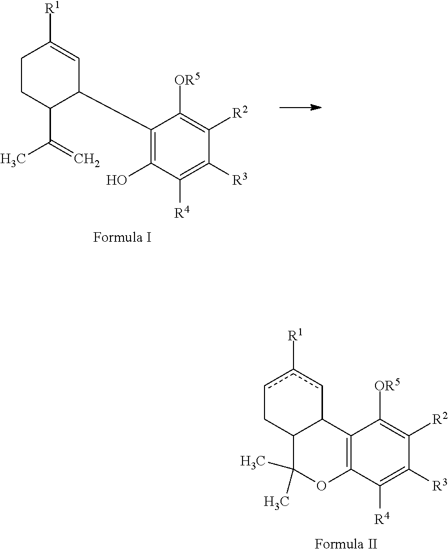

wherein

R¹ is a C₁₋₃ alkyl group, optionally substituted with one or more substituents;

R² and R⁴ each independently is H, halide or —CO₂R⁶, where R⁶ is H or a hydrocarbon having one or more substituents;

R³ is C₁₋₁₀ alkyl group, optionally substituted with one or more substituents;

R⁵ is H or an alcohol protecting group; and

[symbol] is a single or a double bond, provided that one of the [symbol] is a single bond.

In an embodiment, the specification relates to a process wherein a compound of Formula Ia is reacted to form a compound of Formula IIa

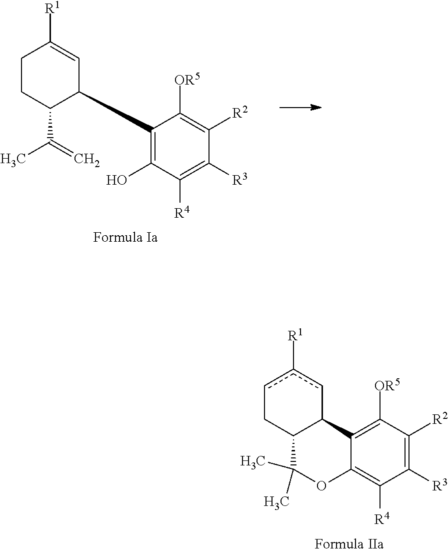

wherein

R¹ is a —CH₃ or —CH₂OH; and

R³ is C₃₋₇ alkyl group, optionally substituted with one or more substituents.

In another aspect, the specification relates to a process for preparation of Δ⁹-tetrahydrocannabinol (Δ⁹-THC) or a derivative thereof, the process having the step of reacting cannabidiol (CBD) or a derivative thereof, in a solvent, in the presence of a solid supported acid catalyst to form Δ⁹-tetrahydrocannabinol (Δ⁹-THC) or a derivative thereof.

In another further aspect, the specification relates to a process for preparation of Δ⁸-tetrahydrocannabinol (Δ⁸-THC) or a derivative thereof, the process having the step of reacting cannabidiol (CBD) or a derivative thereof, in a solvent, in the presence of a solid supported acid catalyst to form Δ⁸-tetrahydrocannabinol (Δ⁸-THC) or a derivative thereof.

### BRIEF DESCRIPTION OF THE DRAWINGS

Reference will now be made, by way of example, to the accompanying drawings which show example embodiments of the present application, and in which:

FIG. 1 is a simplified diagram of a first embodiment of an apparatus in accordance with the specification that is used to carry out the method disclosed herein;

FIG. 2 is a simplified diagram of the first embodiment apparatus in accordance with the specification and is similar to FIG. 2, but with the solid supported acid catalyst in place in the vertical column as retained in place by the filter;

FIG. 3 is a simplified diagram similar to FIG. 3, but with the CBD solution being poured into the vertical column through the top opening;

FIG. 4 is a simplified diagram similar to FIG. 4, with the CBD solution still being poured into the vertical column through the top opening and with the CBD solution flowing through the solid support structure and reacting with the solid support acidic catalyst;

FIG. 5 is a simplified diagram similar to FIG. 5, with the CBD solution still being poured into the vertical column through the top opening and with the CBD solution flowing through the solid support structure and reacting with the acidic catalyst, and also showing the reacted solution;

FIG. 6 is a simplified diagram of a second embodiment apparatus in accordance with the specification that is used to carry out the method disclosed herein;

FIG. 7 is a simplified diagram of the second embodiment apparatus in accordance with the specification and is similar to FIG. 7, but with the CBD solution added to the reaction vessel through the inlet and residing inside the reaction vessel;

FIG. 8 is a simplified diagram of the second embodiment apparatus in accordance with the specification and is similar to FIG. 8, but with a solid support acid catalyst being added to the CBD solution through the reaction vessel inlet;

FIG. 9 is a simplified diagram of the second embodiment apparatus in accordance with the specification and is similar to FIG. 9, but with the apparatus sealed with a stopper and stirred using the stirrer hotplate, such that the solid support acid catalyst is suspended within the CBD solution;

FIG. 10 is a simplified diagram of the second embodiment apparatus in accordance with the specification and is similar to FIG. 10, but the reaction has allowed to stir for a predetermined amount of time and now shows the reacted solution with suspended solid support acid catalyst and is uncapped;

FIG. 11 is a simplified diagram of the second embodiment apparatus in accordance with the specification and is similar to FIG. 11, but the reacted solution is being filtered to remove the solid support acid catalyst;

FIG. 12 is a reaction diagram of the conversion of CBD and its congeners to Δ⁹-THC;

FIG. 13 is a reaction diagram of the conversion of Δ⁹-THC to Δ⁸-THC; and,

FIG. 14 is a simplified diagram of a third embodiment apparatus in accordance with the specification that is used to carry out the method disclosed herein.

Similar reference numerals may have been used in different figures to denote similar components.

### DESCRIPTION

In one aspect, the specification relates to a process for preparation of a compound of Formula II, the process having the step of:

reacting a compound of Formula I, in a solvent, in the presence of a solid supported acid catalyst to form the compound of Formula II

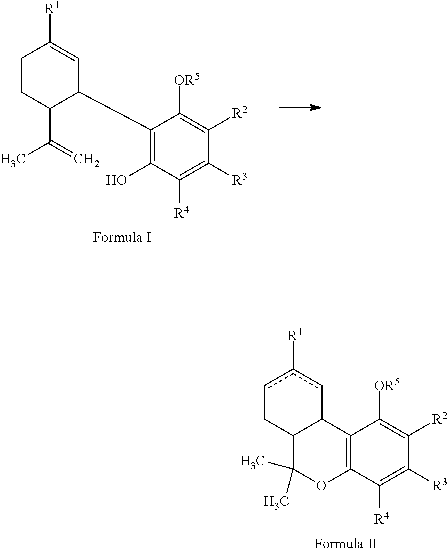

wherein

R¹ is a C₁₋₃ alkyl group, optionally substituted with one or more substituents;

R² and R⁴ each independently is H, halide or —CO₂R⁶, where R⁶ is H or a hydrocarbon having one or more substituents;

R³ is C₁₋₁₀ alkyl group, optionally substituted with one or more substituents;

R⁵ is H or an alcohol protecting group; and

[symbol] is a single or a double bond, provided that one of the [symbol] is a single bond.

The term alkyl group is not particularly limited and should be known to a person of skill in the art. The length of the alkyl group can vary depending upon and can be determined based on non-inventive routine experimentation by a person of skill in the art.

For exemplary purpose, the term C₁₋₆-alkyl in accordance with the specification is not particularly limited and should be known to a person of skill in the art. The C₁₋₆-alkyl may be, for example, and without limitation, any straight or branched alkyl, for example, methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1,2-dimethylbutyl, 1-ethyl-2-methylpropyl, 1,1,2-trimethylpropyl, 1,1-diethyl-2-methylbutyl, 1,1-dimethylbutyl, 2,2-dimethylbutyl, 2-ethylbutyl, 1,3-dimethylbutyl, 2-methylpentyl or 3-methylpentyl.

The term ‘substituents’ as used herein is not particularly limited and should be known to a person of skill in the art, and can be determined based on non-inventive routine experimentation. The substituents used herein should not interfere with the reaction to prevent the cyclization process disclosed in Scheme 2. In one embodiment, the substituents can be, for example and without limitation, a cyclic or non-cyclic alkyl, cyclic or non-cyclic alkenyl, cyclic or non-cyclic alkynyl, aryl, or heteroaryl, optionally with one or more substituents. In another embodiment, for example and without limitation, the substituent is an alcohol, ether, halide, ether, ester, carboxylic acid, or a carbonyl-functional group.

The term “halide” as used herein is not particularly limited and should be known to a person of skill in the art. In one embodiment, for example and without limitation, the halide is Cl, Br or I.

The term ‘hydrocarbon’ is not particularly limited and should be known to a person of skill in the art. In one embodiment, for example and without limitation, the hydrocarbon is a cyclic or non-cyclic alkyl, cyclic or non-cyclic alkenyl, cyclic or non-cyclic alkynyl or aryl, optionally with one or more substituents.

The term “alcohol protecting group” is not particularly limited and should be known to a person of skill in the art. Examples of suitable protecting groups can be found in the latest edition of Greene and Wats, Protecting Groups in Organic Synthesis. In one embodiment, for example and without limitation, the protecting group is acetyl, benzoyl, benzyl, β-methoxyethoxymethyl ether (MEM), dimethoxytrityl (DMT), methoxytrityl (MMT), trityl, methoxymethyl ether (MOM), p-methoxybenzyl ether (PMB), pivaloyl (Piv), trahydropyranyl (THP), tert-butyloxycarbonyl (BOC), tosyl (Ts) or a silyl based protecting groups, such as, for example and without limitation, trimethylsilyl, tert-butyldimethyl silyl (TBDMS), or tert-butyldiphenyl silyl (TBDPS).

The R¹ in the compound of Formula I, Ia, II or IIa is a C₁₋₃ alkyl group, optionally substituted with one or more substituents. In one embodiment, for example and without limitation, the R¹ is —CH₃ or —CH₂OH. In another embodiment, for example and without limitation, R¹ is —CH₃.

The R² and R⁴, in the compound of Formula I, Ia, II or IIa, each independently is H, halide or —CO₂R⁶, where R⁶ is H or a hydrocarbon having one or more substituents. In one embodiment, for example and without limitation, R² and R⁴ in the compound of Formula I, Ia, II or IIa is H.

The R³ in the compound of Formula I, Ia, II or IIa is a C₁₋₁₀ alkyl group, optionally substituted with one or more substituents. In one embodiment, for example and without limitation, R³ in the compound of Formula I, Ia, II or IIa is a C₃₋₇ alkyl group, optionally substituted with one or more substituents. In another embodiment, for example and without limitation, R³ in the compound of Formula I, Ia, II or ha is propyl, butyl, pentyl, hexyl or heptyl. In another further embodiment, for example and without limitation, R³ in the compound of Formula I, Ia, II or ha is pentyl.

In another aspect, the specification relates to a process for preparation of tetrahydrocannabinol (THC) or a derivative thereof, the process having the step of reacting cannabidiol (CBD) or a derivative thereof, in a solvent, in the presence of a solid supported acid catalyst to form tetrahydrocannabinol (THC) or a derivative thereof. In a further aspect, the specification relates to a process for preparation of Δ⁹-tetrahydrocannabinol (Δ⁹-THC) or a derivative thereof. In another aspect, the specification relates to a process for preparation of Δ⁸-tetrahydrocannabinol (Δ⁸-THC) or a derivative thereof.

The term ‘derivative’ is not particularly limited, and should be known to a person of skill in the art. In one embodiment, for example and without limitation, the pentyl side chain of the aromatic group may be substituted with a longer or shorter alkyl side chain, which can be optionally substituted. For example and without limitation, the pentyl side chain can be substituted by a propyl side chain. In addition, or alternatively, in another embodiment, the aromatic moiety can contain one or more substituents, which can be optionally substituted. In one embodiment, for example and without limitation, the aromatic moiety can be substituted. The substituent on the aromatic moiety is not particularly limited and should be known to a person of skill in the art, or can be determined. In one embodiment, for example and without limitation, the substituent on the aromatic moiety is a carboxylic acid group, an ester group, or a halide.

The process as disclosed herein is carried out by an intramolecular cyclization of cannabidiol (CBD) or a derivative thereof to form a cannabinoid having a heterocyclic ring, and involves a nucleophilic attack of the phenoxy-oxygen on the catalyst activated exo-cyclic alkene. In one embodiment, for example and without limitation, the intramolecular cyclization of cannabidiol (CBD) or a derivative thereof to form a cannabinoid having a heterocyclic ring involves a reaction as shown in Scheme 2, where a compound having structural features of Formula E is converted to a compound having structural features of Formula F.

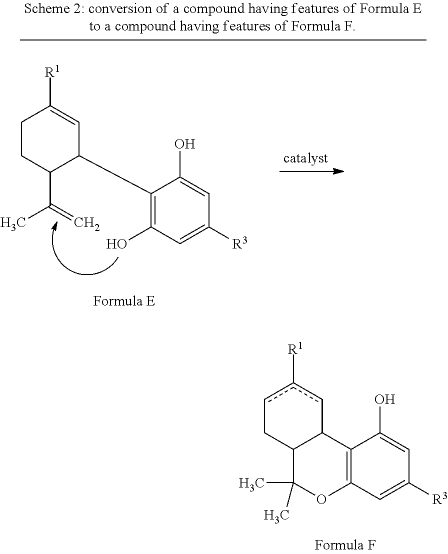

The solvent used for carrying out the reaction is not particularly limited, and should be known to a person of skill in the art, or can be determined. In one embodiment, for example and without limitation, the solvent is an aprotic solvent. In a further embodiment, for example and without limitation, the aprotic solvent is dichloromethane, chloroform, toluene, medium chain triglyceride (MCT), long chain triglyceride (LCT) or supercritical carbon dioxide (CO₂). In another embodiment, the solvent used for the reaction is a medium chain triglyceride (MCT), which can allow the reaction product to be used for subsequent processing, including formulation, and assist with avoiding additional process purification and/or isolation steps.

The term ‘solid support’ is not particularly limited and should be known to a person of skill in the art, or can be determined. Solid supports are used for carrying out solid phase synthesis and are insoluble in the solution phase of the reaction medium. In one embodiment, for example and without limitation, the solid support is a zeolite, a polystyrene based resin, a silicate, celite or a clay material. In another embodiment, for example and without limitation, the solid phase is a smectite-clay. In a further embodiment, for example and without limitation, the solid phase is montmorillonite K 10 (MK10). In another further embodiment, for example and without limitation, the solid phase is Amberlyst 15. In still another embodiment, for example and without limitation, the solid phase is boron trifluoride diethyl etherate (BF₃.EtO₂) on silica.

The term ‘solid support acid catalyst’ is not particularly limited and should be known to a person of skill in the art. In one embodiment, for example and without limitation, the acid in the solid phase acid catalyst can be coupled to the solid phase by a linker or be impregnated on the solid support. In another embodiment, for example and without limitation, the solid support selected has an acidic moiety or functional groups that can function as an acid, for example and without limitation, the solid support has a carboxylic acid or sulfonic acid functional group. The term ‘catalyst’ is not particularly limited and should be known to a person of skill in the art. In general, chemical reactions occur faster in the presence of a catalyst because the catalyst can provide an alternative reaction pathway with a lower activation energy than the non-catalyzed mechanism. In catalyzed mechanisms, the catalyst usually reacts to form a temporary intermediate, which then regenerates the original catalyst in a cyclic process. In one embodiment, for example and without limitation, the solid support acid catalyst is zeolite, an Amberlyst resin, a BF₃ on silica, Celite or a clay material. In another embodiment, for example and without limitation, the solid support acid catalyst is montmorillonite K 10 (MK10) or Amberlyst 15. In still another embodiment, for example and without limitation, the solid support acid catalyst has a Lewis or Bronsted acid associated with the solid support. In a particular embodiment, for example and without limitation, the solid support acid catalyst can avoid use of transition metals. In a further embodiment, for example and without limitation, MK 10 can be used for selective synthesis of Δ⁹-tetrahydrocannabinol (Δ⁹-THC) or a derivative thereof from cannabidiol (CBD). In other further embodiments, for example and without limitation, Amberlyst 15 or BF₃ on silica can be used for selective synthesis of Δ⁸-tetrahydrocannabinol (Δ⁸-THC) or a derivative thereof from cannabidiol (CBD).

The temperature for carrying out the reaction is not particularly limited and will vary depending upon the reagents and conditions, including reaction size. In one embodiment, for example and without limitation, the reaction is carried out at −20° C., −15° C., −10° C., −5° C., 0° C., 5° C., 10° C., 15° C., 20° C., room temperature (around 25° C.) or at an elevated temperature. The elevated temperature is not particularly limited, and can vary based on the solvent system used, and can be determined by a person of skill in the art. In one embodiment, for example and without limitation, the elevated temperature is about 30° C., 35° C., 40° C., 45° C., 50° C., 55° C., 60° C., 65° C., 70° C., 75° C., 80° C., 85°, 90° C. or more.

The time for carrying out the reaction is not particularly limited and can vary depending upon reagents and reaction conditions, and can be determined by a person of skill in the art. In one embodiment, for example and without limitation, the reaction is carried out for 1, 2, 3, 5, 10, 20 or more minutes, to 1, 2, 3, 5, 10, 20 or more hours.

The reaction process is not particularly limited and should be known to a person of skill in the art, or can be determined. In one embodiment, for example and without limitation, the reaction is carried out in a batch reactor or a flow process. In another embodiment, for example and without limitation, the reaction is carried out in a horizontal or coiled glass, or metal column packed reactor. In a further embodiment, the reaction process is carried out as a stirred-batch method over MK10, by flowing a solution of CBD through a column packed with MK10 alone or admixed with a non-reactive processing aid or a column packed with MK10 alone or admixed with a non-reactive processing aid using flow-chemistry techniques.

The work-up after the reaction is not particularly limited and should be known to a person of skill in the art, or can be determined. In one embodiment, for example and without limitation, the work-up process can involve filtration, solvent removal and purification by chromatography, crystallization, distillation or precipitation.

As should be recognized by a person of skill in the art, compounds of Formula I, Ia, II and IIa have at least two stereocenters. The stereocenters are more clearly shown in structures of compounds of Formula Ia and IIa. The specification is not limited to any particular configuration and includes all possible diastereomers. In one embodiment, for example and without limitation, the compound of Formula ha has a cis-configuration. In another embodiment, for example and without limitation, the compound of Formula ha has a trans-configuration. In a further embodiment, for example and without limitation, the compounds of Formula I and II have the stereochemistry as shown in the compounds of Formula Ia and ha, respectively.

In another aspect, the specification discloses an apparatus for and method of converting CBD to at least one other type of cannabinoid of the 113 identified types of cannabinoids. The method and apparatus will now be described in detail with reference to the drawings.

The conversion of cannabidiol (CBD) to cannabinoids such as tetrahydrocannabinols (THCs) and derivatives thereof can be achieved, selectively, in a solvent through the use of a support structure, such as a solid supported structure, following either stirred batch, gravity or pressure-fed column or flow-chemistry methods, or other suitable methods and devices and equipment. The choice of a support structure, solvent and method and other related devices and equipment can be selected to achieve high conversion of CBD to cannabinoids, such as tetrahydrocannabinol derivatives, and to achieve selective derivative formation including selective conversion of CBD to Δ⁹-tetrahydrocannabinol (Δ⁹-THC) or to Δ⁸-tetrahydrocannabinol (Δ⁸-THC), in addition to processing advantages conferred through the employment of the solid support.

In a particular embodiment, the use of solid supported catalysts such as zeolites, Amberlyst resins, clay materials such as montmorrillonite K 10 and other smectite-clays, as well as metal-doped versions of these clays can offer many process chemistry advantages through their use in stirred-batch processing, column (gravity or pressure fed) and flow chemistry processes.

Given the potential applications and demonstrated medicinal value of phytocannabinoids, methods for the selective conversion of CBD to Δ⁸-THC and or Δ⁹-THC are highly desirable. In a particular embodiment, the present disclosure relates to the use of solid-supports such as natural clays, including montmorrillonite K 10 and metal-doped versions thereof, solid support resins, such as Amberlyst 15 or zeolites in stirred-batch, gravity or pressure-fed column and column-flow chemistry processes. The present disclosure relates to the use of these materials and processes in the conversion of CBD to Δ⁸-THC and/or Δ⁹-THC with high yield and selectivity.

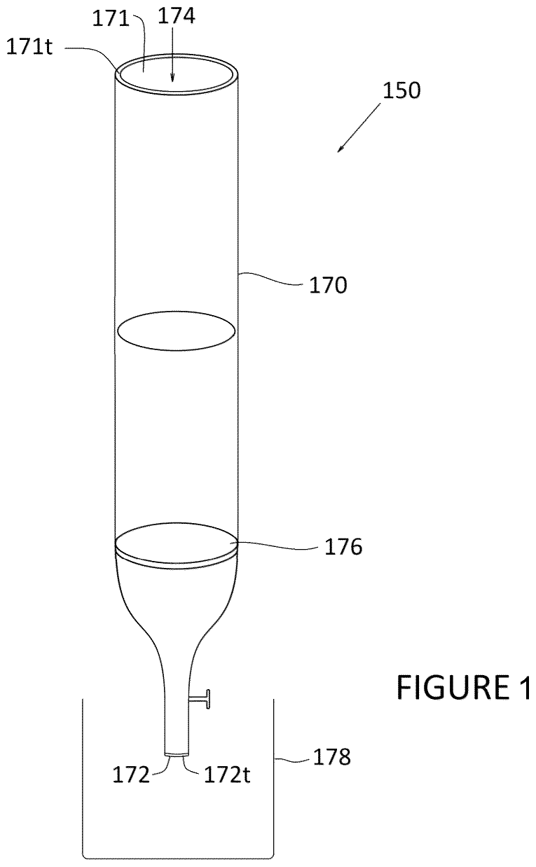

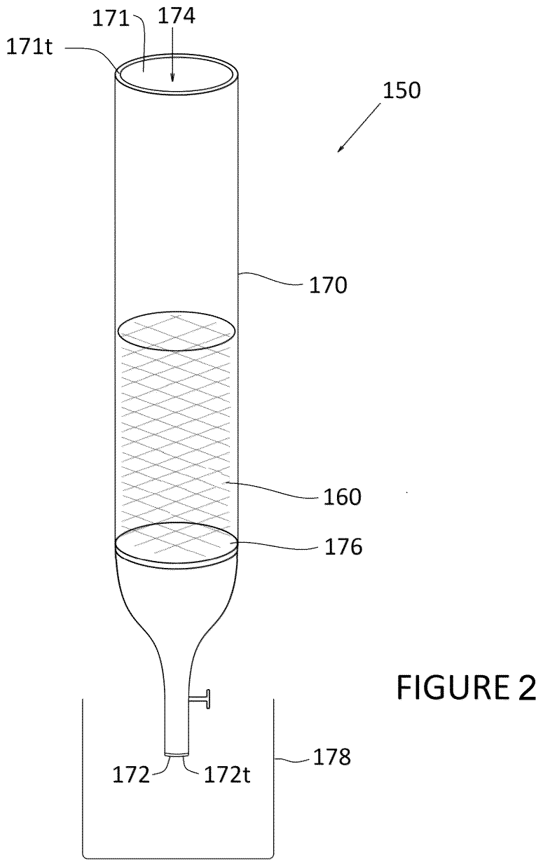

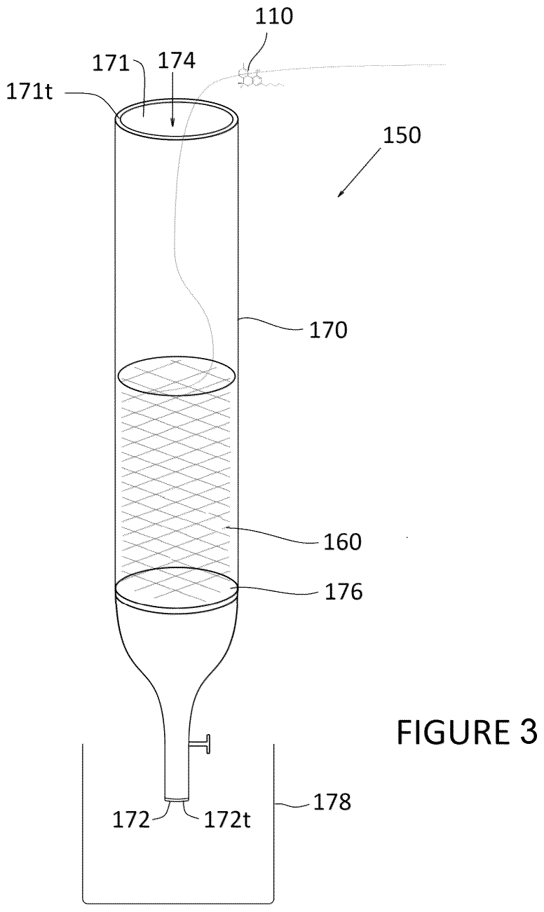

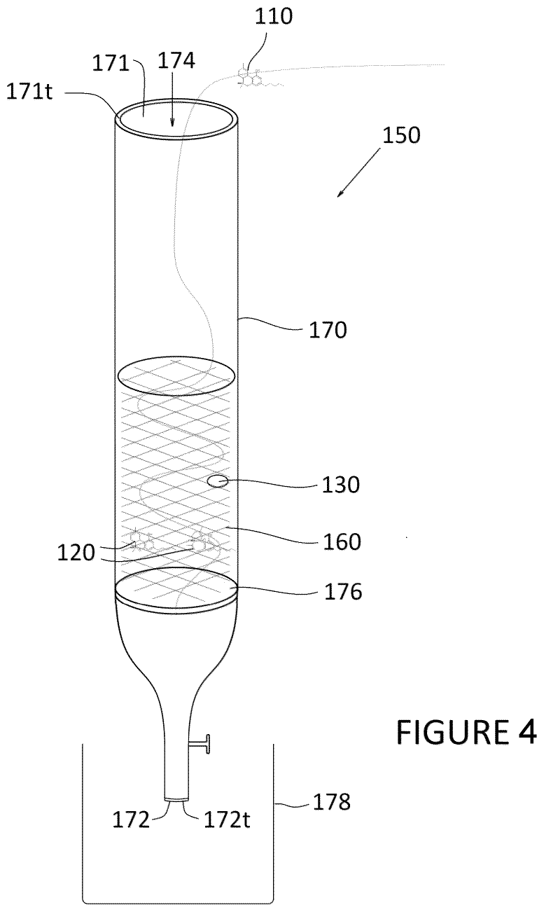

Reference will now be made to FIG. 1 through FIG. 12, which show embodiments of the apparatus for and method of converting CBD and/or CBD derivatives, including CBD-A, to at least one other type of cannabinoid and/or cannabinoid derivative such as THC, according to the present specification.

The first embodiment apparatus and method 100 according to the present specification includes a method of converting CBD 110 to at least one other type of cannabinoid 120 and/or cannabinoid derivative, such as THC 130, and also the apparatus 150 for converting CBD 110 to at least one other type of cannabinoid 120 and/or cannabinoid derivative, such as THC 130. In the following detailed description, for the sake of convenience and for the sake of ease of reading, the term CBD 110 will refer to cannabinoid and/or cannabinoid derivatives, including CBD-A.

In the first embodiment, the apparatus 150 according to the present specification includes a support structure 160 for retaining a catalyst 130. The support structure 160 as illustrated comprises a solid support structure 160. The solid support structure 160 comprises a material having properties of clay material, and more specifically, the porous material comprises a clay material. As presently known, the clay material may be chosen from the group of bentonite, montmorillonite K 10, and other smectite-clays, or may be any other suitable clay material. The catalyst 130, as disclosed herein, is a solid support acid catalyst.

Clay materials such as bentonite, montmorillonite K 10 and other similar clay materials, contain an acidic moiety 140 in their naturally existing state. Accordingly, when clay materials such as bentonite, montmorillonite K 10 and other similar clay materials are used, they act as both the solid support structure 160 and the acidic catalyst 140 together and therefore define a solid supported acid catalyst 165.

It is also contemplated that an acidic catalyst 140 could be added to the clay material by doping with metal or Lewis acids.

As illustrated, the solid support structure 160 is retained in an outer housing 170, specifically a vertically oriented column 170 having an inlet 171 at the top end 171t and an outlet 172 at the bottom end 172b. A through passage 174 connects the top inlet 171 and the bottom outlet 172 in fluid communication with the each other. A filter 176 is disposed in secured relation adjacent the bottom end 172b of the vertically oriented column 170. The filter 176 retains the solid support structure in place in the vertically oriented column 170, and also allows the reacted solution to pass therethrough to thereby be recovered.

In one embodiment, as disclosed herein, the vertically oriented column 170 is cylindrically shaped, or in other words has substantially constant cross-sectional shape from top to bottom, but alternatively could be conically shaped, either with the larger open end at the top and the smaller open and at the bottom, or vice versa.

It is also contemplated that the solid support structure 160 could comprise other materials such as zeolites, Amberlyst resins, among others. It is also contemplated that the support structure 160 could comprise any non-dissolved or phase separated, immiscible, heterogeneous matrix type of material. It is also contemplated that the support structure 160 could comprise a granular material or a fine powder material. Alternatively, the support structure 160 comprises a semi-solid support structure wherein the phase separated material is immiscible within the reaction solvent such as a in a silica gel support structure, or a swollen resin type system.

The method of converting CBD 110 to at least one other type of cannabinoid and/or cannabinoid derivative according to an embodiment of the present specification is described below.

A suitable acidic catalyst 140 is provided. The acidic catalyst 140 may be chosen from at least the group of Lewis acids such as BF₃, BF₃.OEt₂, Ti(OiPr)₄, etc., metal doped catalysts including cations such as Na, Li, Ge, etc. or may be a Bronsted (H⁺) acid, or may be described as being of the general formula MY where M is selected from B, Al, Sc, Ti, Yt, Zr, La, Li, Hf or Zn and Y can be selected from F, Cl, Br, I, trifluoroacetate (triflate) alkoxide and combinations thereof.

In the present embodiment and in order to help facilitate the ready availability of the acidic catalyst 140 in the process, the acidic catalyst 140 may be intrinsically part of the support structure 160, to form the solid support acid catalyst, disclosed herein. Additionally or alternatively, the acidic catalyst 140 may be absorbed into the support structure 160, or in other words is present within the material of the support structure 160, to form the solid support acid catalyst, disclosed herein. Also additionally or alternatively, the acidic catalyst 140 may be adsorbed onto the support structure 160, or in other words is present on the exposed surface as of the support structure 160, to form the solid support acid catalyst, disclosed herein.

The CBD 110 is then introduced into a solvent 112 to create a CBD solution 114. As is presently known, the CBD 110 may be comprised of at least one of a CBD oil, a CBD isolate, a CBD distillate, a CBD liquid, a CBD solid, a CBD vapour, a CBD plant, or other suitable form. In one embodiment, the solvent 112 may be a typical organic solvent 112, for example and without limitation, toluene, tetrahydrofuran, or a halogenated organic solvent, for example and without limitation, chloroform or dichloromethane. The solvent 112 also may be a supercritical fluid. The solvent may also be an oil such as medium chain triglycerides oil or an essential oil. Further, the CBD 110 is then introduced into the solvent 112 via any suitable method such as pouring.

The next step comprises introducing the CBD solution 114 to the solid support acid catalyst 140, typically by flowing the CBD solution 114 past the acidic catalyst 140 on the solid support structure 160 (the solid support acid catalyst). As illustrated, the CBD solution 114 enters the throughpassage 174 of the solid support structure 160 via the top inlet 171, passes over the solid support structure 160 to thereat react with the acidic catalyst 140, and then exits the throughpassage 174 via the bottom outlet 172. As illustrated, the CBD solution 114 may be gravity fed through the throughpassage 174. Also, a pressure differential may be provided between the inlet 171 and the outlet 172 of the throughpassage 174 to cause the flowing of the CBD solution 114 past the acidic catalyst 140 on the solid support structure 160. The pressure differential may have a value and/or range from about 1 psi to about 50 psi. As described, the step of introducing the CBD solution 114 to the acidic catalyst 140 comprises flowing the CBD solution 114 through the solid support structure 160 so as to dynamically contact the acidic catalyst 140 on the support structure 160 (that forms the solid support acid catalyst).

The present method may also further comprise the step of, prior to introducing the CBD solution 114 to the acidic catalyst 140, wetting the solid-support catalyst with the solvent 112.

In order to allow the necessary chemical reaction to proceed properly, the next step is providing sufficient time for the CBD 110 to react with the acidic catalyst 140 to create at least one type of cannabinoid in an overall reaction solution 122. In the specific chemical reaction as discussed subsequently, this step comprises providing sufficient time for the CBD 110 to react with the acidic catalyst 140 to create THC.

Additionally or alternatively, the method according to the present invention may further comprise the step of stirring the overall reaction solution 122. The step of stirring the overall reaction solution 122 is performed during the step of providing sufficient time for the CBD 110 to react with the acidic catalyst 140 to create at least one type of cannabinoid in the overall reaction solution 122.

The step of providing sufficient time for the CBD 110 to react with the acidic catalyst 140 to create at least one type of cannabinoid in the overall reaction solution 122 comprises, for example and without limitation, providing between about one minute and about twenty-four (24) hours for the CBD 110 to react with the acidic catalyst 140 to create at least one type of cannabinoid in the overall reaction solution 122.

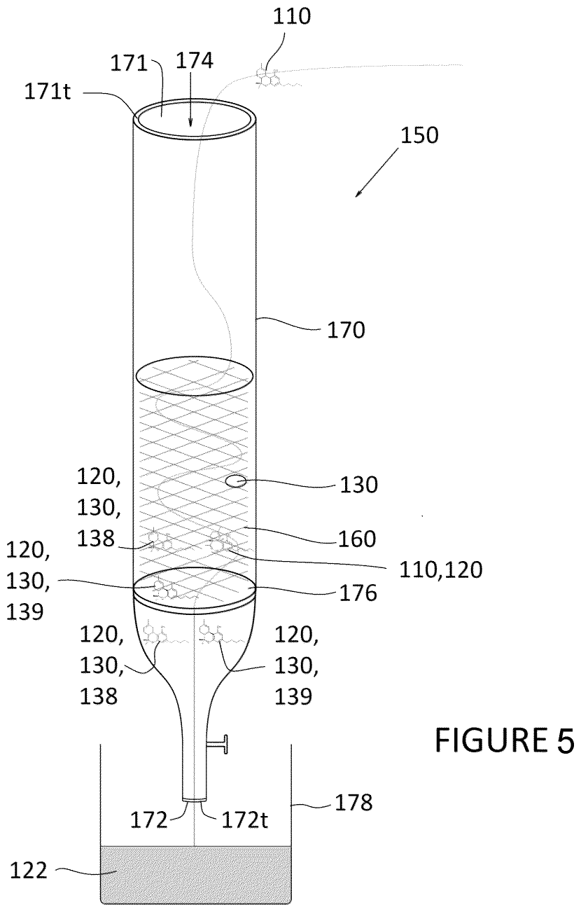

As can be seen in FIG. 5, the reacted solution 122 is captured in a collection vessel 178 for subsequent use.

In order to actually capture the desired yield of at least one type of cannabinoid, such as THC, there is the step of separating the at least one type of cannabinoid from the remainder of the overall reaction solution 122. Such separation can be done by any suitable method such as, for example and without limitation, distillation, evaporation, chromatography, precipitation, recrystallization, and so on. In some embodiments, for example and without limitation, where medium chain triglycerides (MCT) are used for carrying out the reaction, separation of the cannabinoid from the solvent system can be avoided.

If necessary, the method according to an embodiment of the present specification can further comprise the step of, filtering the overall reaction solution 122. This step should be done subsequent to the step of providing sufficient time for the CBD 110 to react with the acidic catalyst 140 to create at least one type of cannabinoid in the overall reaction solution 122. Additionally, this step can be done either before or subsequent to the step of separating the at least one type of cannabinoid from the remainder of the overall reaction solution 122, depending on the specific method and apparatus used.

Optionally, the method according to an embodiment of the present specification can further comprise the step of evaporating the solvent 112. Optionally, the method according to another embodiment of the present specification can further comprise the step of purifying the tetrahydrocannabinol product as necessary.

The present method further comprises the steps of purifying through distillation, evaporation, heating or cooling with or without a plurality of heating and cooling cycles, and with or without filtration with varying degrees of fine particle removal and with or without chemical filtration including activated carbon to ensure purity of the selected cannabinoids.

Specific examples according to the present specification will now be described.

All technical, scientific terms and acronyms used herein have the same meaning as commonly understood by one of the ordinary skill in the art to which the invention belongs. Methods and materials similar or equivalent to those described herein may be used in the practice or investigation of the present invention, the preferred methods and materials employed are hereby described.

As used herein, CBD refers to cannabidiol; Δ⁹-THC refers to Δ⁹-tetrahydrocannabinol; Δ⁸-THC refers to Δ⁸-tetrahydrocannabinol, and Δ⁸-iso-THC refers to as-iso-tetrahydrocannabinol, the structures of which are reported in Scheme 1.

As used herein, a solid supported acid catalyst 165 refers to a solid material formed as the solid support structure 160 and the catalyst 130 together. The solid supported acid catalyst 165 as disclosed is non-soluble in the reaction media, specifically the CBD/solvent solution. Examples of such a solid supported acid catalyst 165 include but are by no means limited to montmorillonite K 10 and other clay materials, metal-doped clays, zeolites, polymeric resins including Amberlyst 15. Solid-supports containing similar functional groups may also be substituted as appropriate, aspects that will be understood by practitioners skilled in the art.

Described herein are methods and protocols for the conversion of CBD to Δ⁹-THC or to Δ⁸-THC. The reaction time and temperatures may be varied somewhat leading to products of varying yield and selectivity, aspects that will be understood by practitioners skilled in the art.

Specifically, the present disclosure relates to the preparation of Δ⁹-THC from CBD consisting of: production of a solution of CBD in a suitable solvent, such as solvent 112, exposure to this solution with a solid supported acid catalyst 165 for a particular length of time and at a given temperature, separation of the solid supported acid catalyst 165, removal of the organic solvent 112 and purification of the resulting Δ⁹-THC as necessary.

Specifically, the present disclosure relates to the preparation of Δ⁸-THC from CBD consisting of: production of a solution of CBD in a suitable solvent, such as solvent 112, contact of this solution with a solid supported acid catalyst 165 for a particular length of time and at a given temperature, separation of the solid supported acid catalyst 165, removal of the organic solvent and purification of the resulting Δ⁸-THC as necessary.

Hereunder, the specification is described employing representative non-limiting examples.

### Example 1: Conversion of CBD to Δ⁹-THC

A one (1) mL solution of 25 mg/mL CBD 110 in chloroform is loaded onto the solid supported acid catalyst 165, which is a 500 mg vertical column of montmorillonite K10, and is allowed to remain in contact with the solid supported catalyst for a period between one and two minutes and is then eluted with a suitable organic solvent, such as solvent 112 over a period of between one and two minutes. In the specifically described method, evaporation of the solvent gave a 20:1 mixture of Δ⁹-THC:CBD in 98% yield (by mass balance measurement) and 95% purity, determined by a suitable method such as LC, GC or ¹H NMR analysis. It must be understood that these various amounts and measurements of volume, concentration, time, yield, and purity, are cited for this particular experiment only and may be quite different in other experiments and in commercial production.

In the example described above, the CBD solution 114 is passed through a vertical column 170 under gravity or slight positive pressure (flash chromatography) containing montmorillonite K 10 as the solid support structure 160. In another embodiment, the montmorillonite K 10 is blended with a second inert solid-support material as a flow aid. Inert solid-support materials may be selected from various commercial grades of silica gel, alumina, sand or celite.

It is also contemplated that the present reaction could take place in a pressurized vessel, from a pressure slightly above ambient atmospheric pressure to perhaps 2000 PSI, or even significantly more, in an ultrahigh-pressure liquid chromatography system.

In other embodiments, the reaction is conducted under stirred-batch conditions, consisting of stirring the solution of CBD containing montmorillonite K 10 at a set temperature, for a given time.

In other embodiments, the reaction may be conducted under stirred-batch conditions, consisting of stirring the solution of CBD over the solid supported acid catalyst, such as montmorillonite K 10 and a second inert solid-support material, or any other suitable material, as a flow aid, at a set temperature or temperature gradient for a given time. Inert supports may be selected from various commercial grades of silica gel or alumina.

In other embodiments, the reaction is conducted under column-flow conditions, consisting of pumping the solution of CBD through a sealed cartridge or column containing montmorillonite K 10, at a set temperature, or within a temperature range, for a given time.

In other embodiments, the reaction is conducted under column-flow conditions, consisting of stirring the solution of CBD containing montmorillonite K 10 and a second inert solid-support material as a flow aid, in a sealed cartridge, at a set temperature or range of temperatures for a given time or within specific time ranges. Inert supports may be selected from various commercial grades of silica gel or alumina.

In the variations of example 1 described above, the reaction may be conducted under or using a flow of an inert gas such as nitrogen or argon.

In the variations of example 1 described above, the organic solution containing the desired product(s) may be filtered through a plug or short column containing a non-soluble weak base to ensure neutrality of the remaining constituents.

In the variations of example 1 described above, the product Δ⁹-THC may be isolated by removal of the organic solvent from the filtered stirred-batch or eluant from the vertical-column or column-flow method on a rotary evaporator, or by any other suitable method or means. The product may be used as obtained or eluted by column chromatography or distillation.

Various embodiments are described above, although it is recognized and understood that modifications may be made to these, and the claims appended herein are intended to cover all such modifications using solid supported acid catalysts 165 that fall within the scope of the invention.

### Example 2: Conversion of CBD to Δ⁸-THC

A one (1) mL solution of 25 mg/mL solution of CBD in dichloromethane is stirred with Amberlyst 15 (20% by weight relative to CBD) at room temperature for 18 hours before filtering off the solid support acid catalyst; evaporation of the organic solvent 112 gives a resin containing Δ⁸-THC in 95% yield (by mass balance measurement) and 75% purity, as determined by a suitable method such as LC, GC or ¹H NMR analysis.

In the example described above, the reaction is conducted under stirred-batch conditions, consisting of stirring the solution of CBD containing Amberlyst 15 at a set temperature, for a given time.

In other embodiments, the solution of CBD is passed through a vertical column under gravity or slight positive pressure (flash chromatography) containing Amberlyst 15. In another embodiment, the Amberlyst 15 is blended with a second inert solid-support material as a flow aid. Inert supports may be selected from various commercial grades of silica gel or alumina.

In other embodiments, the reaction is conducted under stirred-batch conditions, consisting of stirring the solution of CBD containing Amberlyst 15 and a second inert solid-support material as a flow aid, at a set temperature, for a given time. Inert supports may be selected from various commercial grades of silica gel or alumina.

In other embodiments, the reaction is conducted under column-flow conditions, consisting of pumping the solution of CBD through a sealed cartridge or column containing Amberlyst 15, at a set temperature, for a given time.

In other embodiments, the reaction is conducted under column-flow conditions, consisting of stirring the solution of CBD containing Amberlyst 15 and a second inert solid-support material as a flow aid, in a sealed cartridge, at a set temperature, for a given time. Inert supports may be selected from various commercial grades of silica gel or alumina.

In the variations of example 2 described above, the reaction may be conducted under or using a flow of an inert gas such as nitrogen or argon.

In the variations of example 2 described above, the organic solution containing the desired product(s) may be filtered through a plug or short column containing a non-soluble weak base to ensure neutrality of the remaining constituents.

In the variations of example 2 described above, the product Δ⁸-THC may be isolated by removal of the organic solvent 112 from the filtered stirred-batch or eluant from the vertical-column or column-flow method on a rotary evaporator. The product may be used as obtained or eluted by column chromatography or distillation.

Various embodiments are described above, although it is recognized and understood that modifications may be made to these, and the claims appended herein are intended to cover all such modifications using solid supported acid catalysts 165 that fall within the scope of the specification.

According to another aspect of this invention, the solid supported acid catalyst 165 or functionalized resin may be reused after elution of the reaction mixture.

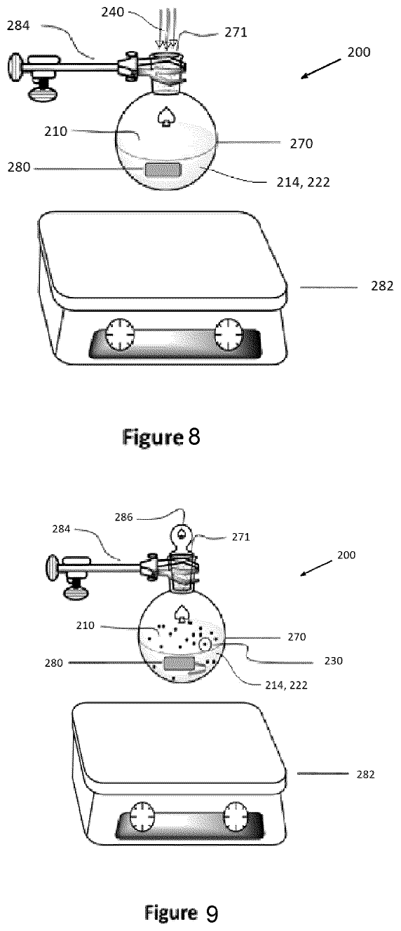

Reference will now be made to FIG. 8 through FIG. 11, which show a second embodiment of the apparatus for and method of converting CBD and/or CBD derivatives, including CBD-A, to at least one other type of cannabinoid and/or cannabinoid derivative such as THC, according to the present specification.

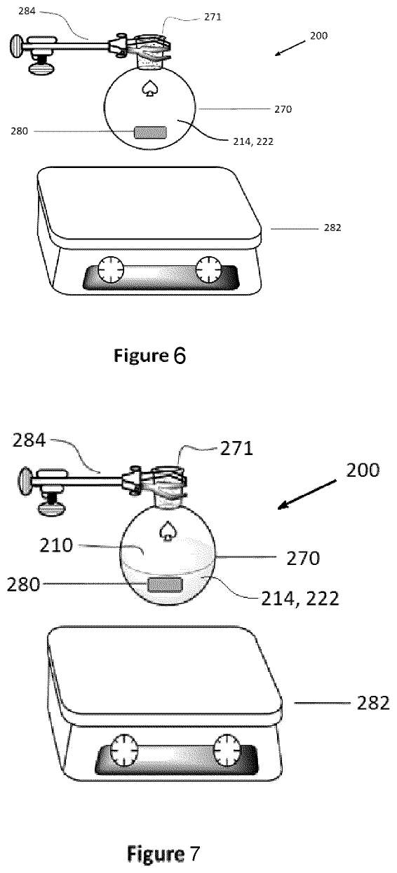

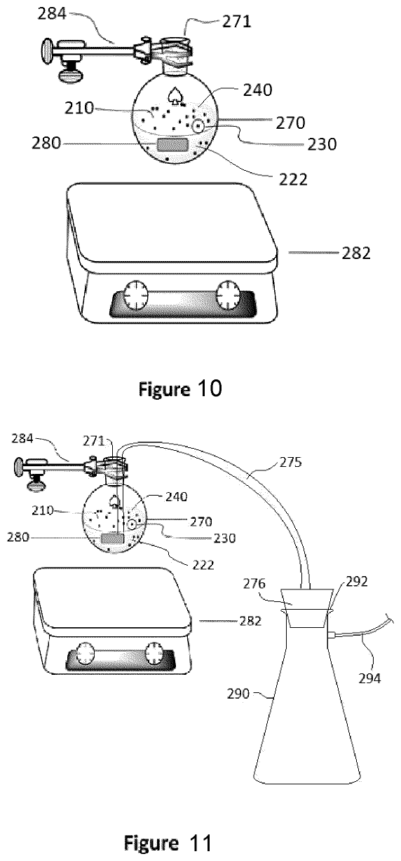

In the second embodiment apparatus and method 200, FIG. 6 shows a reaction vessel 270 containing a magnetic stir bar 280 and help by clamp 284 over a stirrer hotplate 282. FIG. 7 shows the CBD solution 214 added to the reaction vessel 270 through the inlet 271 and residing inside the reaction vessel 270. FIG. 8 shows a non-soluble acidic catalyst 240 (a solid supported acid catalyst) being added to the CBD solution 214 through the reaction vessel inlet 271. FIG. 9 shows the apparatus 250 sealed with a stopper 286. The acidic catalyst 240 is suspended within the CBD solution 214 to form a reaction solution 222. The reacted solution 222 is being stirred using the stirrer hotplate 282, FIG. 10 shows the reaction has been allowed to stir for a predetermined amount of time and now shows the reacted solution 222 with suspended acidic catalyst 240. Also, the stopper 286 has been removed. FIG. 11 shows the reacted solution 222 being suctioned into a collection flask 290 having a filter 276 engaged in sealed relation on the top mouth 292 of the collection flask 290. Air is drawn from the collection flask 290 by an air pump (not shown) through air suction hose 294, which in turn suctions the reaction solution 222 from the reaction vessel 270 through the liquid suction hose 275. The reaction solution 222 is being filtered by filter 276 as it is suctioned into the collection flask 290 to remove the non-soluble acidic catalyst 240.

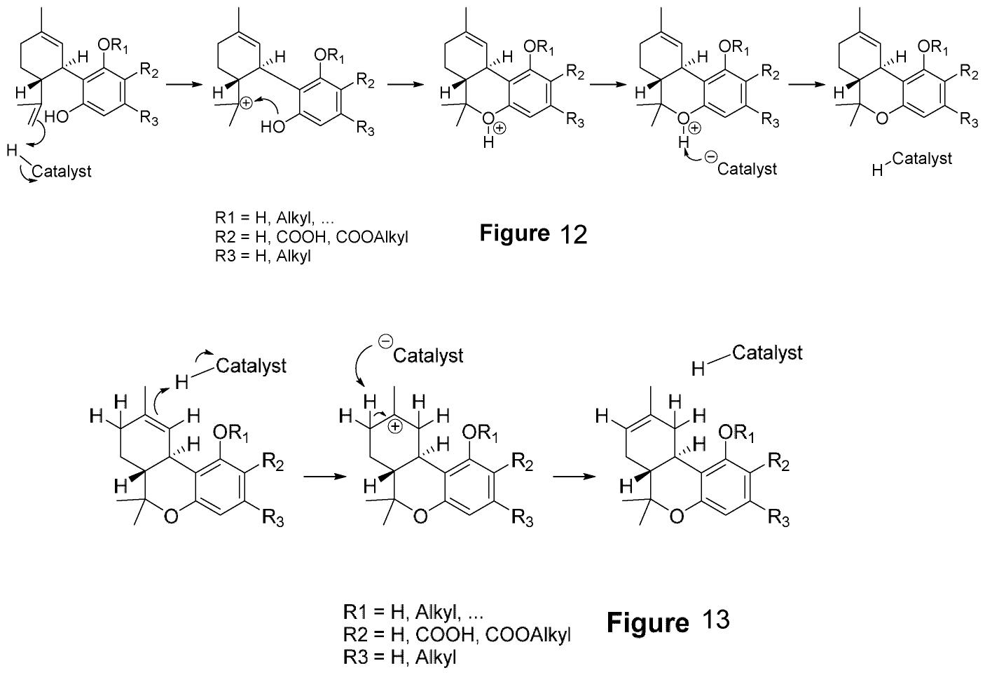

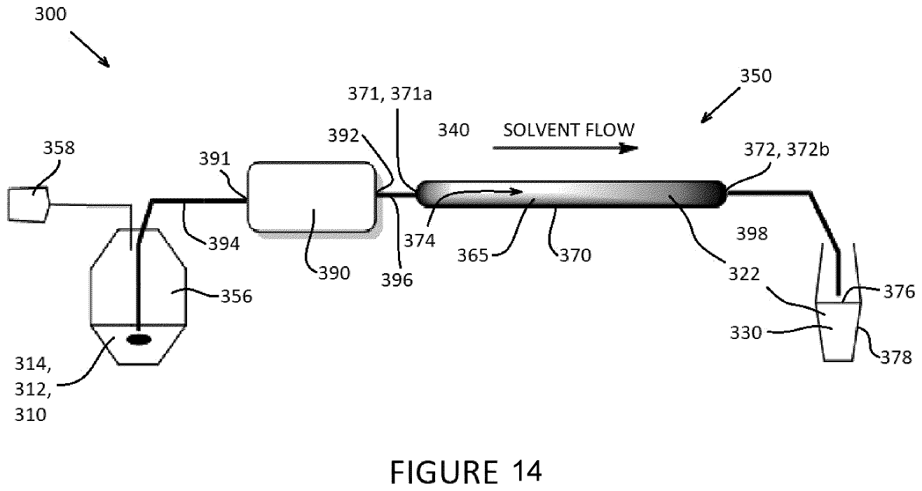

Reference will now be made to FIGS. 12 and 13, which are applicable to embodiments disclosed in the specification. More specifically, FIG. 12 is a reaction diagram of the conversion of CBD to Δ⁹-THC and its congeners, and FIG. 14 is a reaction diagram of the conversion of Δ⁹-THC to Δ⁸-THC.

Reference will now be made to FIG. 14, which shows a third embodiment of the apparatus for and method of converting CBD and/or CBD derivatives, including CBD-A, to at least one other type of cannabinoid and/or cannabinoid derivative such as THC, according to the present specification. More specifically, in the third embodiment apparatus and method 300, in FIG. 14, the apparatus 350 is oriented generally horizontally. Accordingly, gravity cannot be relied on to cause the flow of the reaction solution 322 through the apparatus 350. Instead, a pump 390 is employed as will now be described.

A starting vessel 356 contains the CBD solution 314 (the combination of the CBD 310 and the solvent 312). The starting vessel 356 may be sealed off during the conversion operation and the ambient air purged from the starting vessel 356 by a purge pump 358. The pump 390 is connected in fluid communication at its inlet end 391 with the starting vessel 356 via suction tube 394 and is connected in fluid communication at its outlet end 392 with a horizontally oriented cylinder 370 via an inlet 371 at its inlet end 371a via delivery tube 396. The horizontally oriented cylinder 370 operatively retains the solid supported catalyst 365 (the solid supported acid catalyst) within an internal throughpassage 374, and is connected in fluid communication via an outlet 372 at its outlet end 372b with a product collection vessel 378 via delivery tube 398. A filter 376 is disposed in secured relation within the product collection vessel 378.

During the conversion operation, the pump 390 suctions the CBD solution 314 from the starting vessel 356 and pumps the CBD solution 314 through the horizontally oriented cylinder 370 and into the product collection vessel 378. The acidic catalyst 340 that is an integral part of the solid supported catalyst 365 React with the CBD 310 (and/or CBD derivatives) in the CBD solution 314 to create the cannabinoid and/or cannabinoid derivatives such as THC 330.

It should also be understood that for any embodiment where it is suitable, there could additionally be the step of purging the reaction vessel, such as a reaction flask or reaction column, or the like, with nitrogen or with an inert gas prior to the reaction.

Other variations of the above principles will be apparent to those who are knowledgeable in the field of the invention, and such variations are considered to be within the scope of the present invention. Further, other modifications and alterations may be used in the design and manufacture of the present invention without departing from the spirit and scope of the accompanying claims.

### Example 3: Conversion of CBD to THC

Two different processes, consisting of a stirred batch process or a catalyst column reactor, were used. A stirred batch process is essentially a typical organic chemistry reaction. The process involves dissolving CBD in a solvent, adding the catalyst, and stirring for a certain amount of time, temperature, etc. For the catalyst column reactor, the CBD is dissolved in a solvent and passed through a certain amount of catalyst which has been pre-loaded on a column. This is essentially the same concept as a continuous flow reactor.

General Steps for a Stirred Batch Process:

1. CBD is dissolved in a solvent and placed in a reaction vessel with a mechanical stirring/agitating device.

2. The solid catalyst is added directly to the reaction mixture, and the mixture is continuously stirred/agitated to ensure that the catalyst is homogenously distributed within the mixture for the duration of the reaction.

2 (a). Alternatively, the reaction may be cooled or heated prior to adding the catalyst.

2 (b). The reaction may be performed under inert atmosphere, but this is not necessary.

3. The reaction is allowed to stir for a certain amount of time.

4. The solid catalyst is removed from solution via filtering the reaction mixture, or centrifuging the mixture and decanting the supernatant.

4 (a). If desired, the reaction can be filtered through a mildly basic material (e.g. NaHCO₃) that ensures that any trace acid is quenched

5. The solvent is evaporated, leaving a clear, near colourless cannabinoid resin.

5 (a). Alternatively, if the solvent is very high boiling, e.g. MCT oil, it is not removed.

6. If desired, the purity of the cannabinoids can be increased by any number of standard techniques including chromatography, distillation, sublimation, etc.

In the following trials, reactions were performed at a concentration of 25 mg/mL in the solvent. Catalyst loadings are given as a weight percentage relative to the mass of the starting material. Yields and purity are measured on the crude material obtained after filtration and solvent removal.

| | | | | | HPLC (area %) | NMR Integration |
|---|---|---|---|---|---|---|
| Trial | Solvent | Catalyst | Time | Yield | (Δ⁹:CBD:Δ⁸/iso-Δ⁸) | Δ⁹:CBD:Δ⁸:iso-Δ⁸ |
| 1 | DCM | MK10 | 1 hour | 98% | 42.3:49.8:7.9 | 1:00:1.06:0.0:0.15 |
| | | (10%) | | | | |

| | |
|---|---|
| Comments | Approx. 1:1 mixture of CBD to Δ⁹-THC obtained. 15% of iso-Δ⁸ |
| | formed. Purity of CBD + Δ⁹-THC vs iso-Δ⁸ = 93% |

| | | | | | | |
|---|---|---|---|---|---|---|
| 2 | DCM | MK10 | 18 hour | 96% | 75:7.9:17.1 | 1.00:0.07:0.0:0.15 |
| | | (10%) | | | | |

| | |
|---|---|
| Comments | Almost total consumption of CBD. Amount of iso-Δ⁸ does not increase |
| | vs 1 hr time point. Purity of Δ⁹-THC vs all other cannabinoids = 82%. |
| | Purity of CBD + Δ⁹-THC vs. iso-Δ⁸ = 88% |

| | | | | | | |
|---|---|---|---|---|---|---|
| 3 | DCM | MK10 | 18 hour | 99% | 77.2:0.0:22.8 | 1.00:0.0:0.15:0.16 |
| | | (15%) | | | | |

| | |
|---|---|
| Comments | Total consumption of CBD. Evidence that Δ⁹-THC is being converted to |
| | Δ⁸ THC at higher loadings. Purity of Δ⁹-THC vs all other cannabinoids = 76%. |

| | | | | | | |
|---|---|---|---|---|---|---|
| 4 | CHCl₃ | MK10 | 18 hour | 95% | 83.2:1.0:15.8 | 1.00:0.0:0.17:0.05 |
| | | (15%) | | | | |

| | |
|---|---|
| Comments | Total consumption of CBD. Notably less iso-Δ⁸ formed than in reactions |
| | using DCM. Purity of Δ⁹-THC vs all other cannabinoids = 82% |

| | | | | | | |
|---|---|---|---|---|---|---|
| 5 | PhMe | MK10 | 18 hour | 94% | 77.6:10.7:11.7 | 1.00:0.11:0.04:0.12 |
| | | (200%) | | | | |

| | |
|---|---|
| Comments | Toluene (and other solvents) works. Can use higher catalyst loading. |
| | Purity of Δ⁹-THC vs all other cannabinoids = 75%. Purity of CBD + Δ⁹- |
| | THC vs. iso-Δ⁸ + Δ⁸ = 87% |

| | | | | | HPLC (area %) | NMR Integration |
|---|---|---|---|---|---|---|
| Trial | Solvent | Catalyst* | Time | Yield | (Δ⁹:CBD:Δ⁸/iso-Δ⁸) | Δ⁹:CBD:Δ⁸:iso-Δ⁸ |
| 6 | DCM | MK10- | 18 hour | 96% | 72.9:0.0:27.1 | 1.00:0.0:0.22:0.20 |
| | | ZnCl₂ | | | | |
| | | (30%) | | | | |

| Comments | *A batch of ZnCl₂ doped MK10 was prepared by adding 3.25 mL of | | | | | |
|---|---|---|---|---|---|---|
| | ZnCl₂ (1.0M in Et₂O) to 420 mg of MK10 and evaporating to dryness. | | | | | |
| | This modified catalyst also works, and demonstrates that Lewis acid | | | | | |
| | doped MK10 can still catalyze the desired reaction, opening potential | | | | | |
| | for multi-functional catalysts. | | | | | |
| | | | | | HPLC (area %) | NMR Integration |
| Trial | Solvent | Catalyst* | Time | Yield | (Δ⁹:Δ⁸) | Δ⁹:CBD:Δ⁸:iso-Δ⁸ |
| 7 | MCT oil | MK10 | 14 hour | n/a | 22.9:67.0 | n/a |
| | | (100%) | | | | |

| | |
|---|---|
| Comments | This reaction was performed at 80° C., and only analyzed by HPLC, as |
| | NMR predominantly shows MCT oil, which is not easily removed. HPLC |
| | demonstrates the sample to be a 90% pure mixture of approx. 1:3 Δ⁹:Δ⁸-THC. |

| | | | | | | |
|---|---|---|---|---|---|---|
| 8 | MCT oil | MK10 | 14 hour | n/a | 37.7:38.4 | n/a |
| | | (50%) | | | | |

| | |
|---|---|
| Comments | This reaction was performed at 80° C., and only analyzed by HPLC, as |
| | NMR predominantly shows MCT oil, which is not easily removed. HPLC |
| | demonstrates the sample to be a 76% pure mixture of approx. 1:1 Δ⁹:Δ⁸-THC. |

| | | | | | HPLC (area %) | |
|---|---|---|---|---|---|---|
| Trial | Solvent | Catalyst* | Time | Yield | (Δ⁹:Δ⁸) | NMR Integration |
| 9 | DCM | BF₃ on SiO₂ | 18 hour | 96% | n/a | 1.00 Δ⁸-THC to |
| | | (20%) | | | | 0.2 unidentified |
| | | | | | | cannabinoid, |
| | | | | | | 0.03 of iso-Δ⁸ |

| | |
|---|---|
| Comments | Sample is 81% pure Δ⁸-THC by NMR No residual CBD, but an |
| | unidentified cannabinoid product is present in the sample. |

Potential Benefits of a Stirred Batch Process Using Solid Support Acid Catalysts

Aqueous-organic work-up is avoided, lessening material cost and time, and also reducing the potential for losing desired product through excessive manipulations of the product (e.g. washing organic phase, drying organic phase over drying agent).

The catalyst is easily measured and manipulated and does not require air or moisture sensitive operations.

Catalyst is easily recovered and can be used again, if desired.

The catalyst does not decompose over time, unlike other catalysts such as BF₃-Et₂O used in prior patents.

Solid supported catalysts are usually very inexpensive.

The catalysts can be used in more unusual solvents, such as MCT oil.

Able to prepare selectively prepare Δ⁸ or Δ⁹ THC with good purity of Δ⁹ and Δ⁸-THC product.

General Steps for a Catalyst Column Reactor

1). CBD is dissolved in a reaction solvent.

2). The CBD solution is passed through a column containing a certain amount of catalyst solid phase.

2a). The amount of catalyst and flow rate of reactant solution can be varied to obtain different ratios of reactants:products.

2b). The catalyst may contain a certain percentage of SiO₂ gel or other non-reactive fillers to facilitate solvent flow around the catalyst.

2c). The catalyst layer may be preceded by non-reactive solids that help protect the catalyst layer from physical perturbation, or may serve other purposes (e.g. MgSO₄ can be added on top of the catalyst layer to help ensure that the reaction solvent is dry, but this is not necessary); the catalyst layer may be proceeded by non-reactive solids that help protect the catalyst layer from physical perturbation and/or to ensure the pH neutrality of the eluent (e.g. NaHCO₃).

2d). The residence time of the reaction mixture on the column can be varied.

2e). The temperature of the reaction apparatus can be varied.

3). The column is washed with the reaction solvent to ensure complete removal of the reactants and products.

4). The solvent is evaporated, leaving a clear, near colourless cannabinoid resin.

5). If desired, the purity of the cannabinoids can be increased by any number of standard techniques including chromatography, distillation, sublimation, etc.

6). If desired, the column may be reused in future reactions.

In the following trials, reactions were performed at a concentration of 25 mg/mL in the solvent. The amount of catalyst used in the reactor is relative to the amount of starting reactant used (e.g. 10× mass of CBD). Time includes the washing the product off the column reactor with an equal volume of solvent. Yields and purity are measured on the crude material obtained after solvent removal.

| | | | Time | | HPLC (area %) | NMR Integration |
|---|---|---|---|---|---|---|
| Trial | Solvent | Catalyst* | (min.) | Yield | (Δ⁹:CBD:Δ⁸/iso-Δ⁸) | Δ⁹:CBD:Δ⁸:iso-Δ⁸ |
| 10 | CHCl₃ | MK10 | 3 | 99% | 72.9:21.4:5.6 | 1.00:0.29:0.0:0.04 |
| | | (10x) | | | | |

| Comments | Sample is almost entirely starting material (CBD) and desired | | | | | |
|---|---|---|---|---|---|---|
| | product (Δ⁹-THC) in approximately an approximately 2:9 ratio | | | | | |
| | with only trace amounts of other impurities. | | | | | |
| | | | | | HPLC (area %) | |
| Trial | Solvent | Catalyst* | Time | Yield | (Δ⁹:Δ⁸) | NMR Integration |
| 11 | CHCl3 | MK10 | 3.3 minutes | 98% | 84.8:8.7:6.5 | 1.00:0.06:0.02:0.04 |
| | | (20x) | | | | |

| | |
|---|---|
| Comments | Starting material is almost completely consumed. Very trace amount |
| | of other isomers formed. The sample is essentially a 95% pure 20:1 |
| | mixture of Δ⁹-THC:CBD, or could be considered 89% pure Δ⁹-THC. |

Potential Benefits of a Catalyst Column Reactor Using Method Disclosed Herein

Aqueous-organic work-up is avoided, lessening material cost and time, and also reducing the potential for losing desired product through excessive manipulations of the product (e.g. washing organic phase, drying organic phase over drying agent)

The catalyst is easily measured and manipulated and does not require air or moisture sensitive operations. The catalyst does not decompose over time, unlike other catalysts such as BF₃-Et₂O.

Reaction times can be much lower than reported in literature solution methods.

The reaction apparatus can be reused in future reactions.

Solid supported catalysts are usually very inexpensive.

Purity of the crude Δ⁹-THC obtained using this method can be greater than other methods known in the art.

Certain adaptations and modifications of the described embodiments can be made. Therefore, the above discussed embodiments are considered to be illustrative and not restrictive.

1. A process for preparation of a compound of Formula II, comprising:
reacting a compound of Formula I, in a solvent, in the presence of a solid supported acid catalyst to form the compound of Formula II 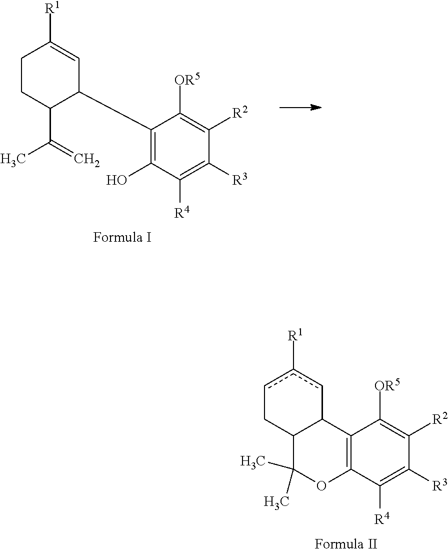
wherein
R¹ is a C₁₋₃ alkyl group, optionally substituted with one or more substituents;
R² and R⁴ each independently is H, halide or —CO₂R⁶, where R⁶ is H or a hydrocarbon having one or more substituents;
R³ is C₁₋₁₀ alkyl group, optionally substituted with one or more substituents;
R⁵ is H or an alcohol protecting group; and [symbol]
is a single or a double bond, provided that one of the [symbol] is a single bond.

2. A process of claim 1, wherein a compound of Formula Ia is reacted to form a compound of Formula IIa 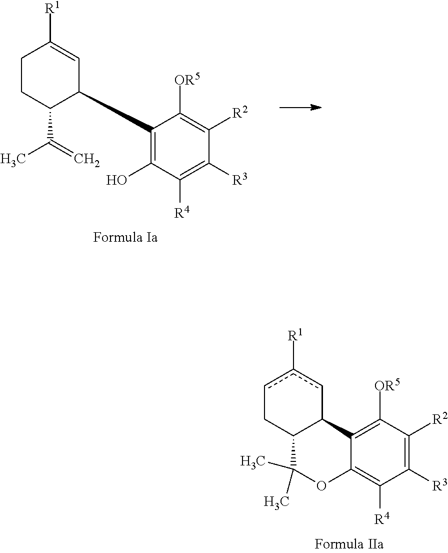
wherein
R¹ is a —CH₃ or —CH₂OH; and
R³ is C₃₋₇ alkyl group, optionally substituted with one or more substituents.

3. The process of claim 2, wherein the compound of Formula IIa is Δ9-tetrahydrocannabinol (Δ9-THC).

4. The process of claim 2, wherein the compound of Formula IIa is Δ8-tetrahydrocannabinol (Δ8-THC).

5. The process of claim 1, wherein the solvent is an aprotic solvent.

6. The process of claim 5, wherein the aprotic solvent is dichloromethane, chloroform, toluene or medium chain triglyceride (MCT).

7. The process of claim 6, wherein the solvent is dichloromethane or chloroform.

8. The process of claim 6, wherein the solvent is medium chain triglyceride (MCT).

9. The process of claim 6, wherein the solvent is supercritical carbon dioxide.

10. The process of claim 1, wherein the solid supported acid catalyst is a zeolite, an amberlyst resin, a silicate, celite or a clay material.

11. The process of claim 10, wherein the clay material is a smectite-clay.

12. The process of claim 10, wherein the clay material is montmorillonite K 10 (MK10).

13. The process of claim 10, wherein the amberlyst resin is Amberlyst 15.

14. A process for preparation of Δ9-tetrahydrocannabinol (Δ9-THC) or a derivative thereof, the comprising:
reacting cannabidiol (CBD) or a derivative thereof, in a solvent, in the presence of a solid supported acid catalyst to form Δ9-tetrahydrocannabinol (Δ9-THC) or a derivative thereof.

15. The process of claim 14, wherein the solid supported catalyst is montmorillonite K 10 (MK10).

16. A process for preparation of Δ9-tetrahydrocannabinol (Δ8-THC) or a derivative thereof, the comprising:
reacting cannabidiol (CBD) or a derivative thereof, in a solvent, in the presence of a solid supported acid catalyst to form Δ8-tetrahydrocannabinol (Δ8-THC) or a derivative thereof.

17. The process of claim 16, wherein the solid supported catalyst is Amberlyst 15.

18. The process of claim 14, wherein the solvent is an aprotic solvent.

19. The process of claim 18, wherein the aprotic solvent is dichloromethane, chloroform, toluene or medium chain triglyceride (MCT).

20. The process of claim 18, wherein the solvent is dichloromethane or chloroform.

21. The process of claim 18, wherein the solvent is medium chain triglyceride (MCT).

22. The process according to claim 1, wherein the process is carried out as a batch process.
